(19) United States
(12) Patent Application Publication
(10) Pub. No.: US 2026/0334902 A1
(43) Pub. Date: Oct. 29, 2026

(54) MAMMOGRAPHY APPARATUS INCLUDING IMPROVED RADIATION DETECTOR

(71) Applicant: DRTECH CORPORATION, Seongnam-si Gyeonggi-do (KR)

(72) Inventors: Choul Woo SHIN, Seongnam-si Gyeonggi-do (KR); Jae Dong LEE, Seongnam-si Gyeonggi-do (KR); IL Woong CHOI, Seoul (KR)

(73) Assignee: DRTECH CORPORATION, Seongnam-si Gyeonggi-do (KR)

(21) Appl. No.: 19/700,996

(22) Filed: Jun. 8, 2026

Related U.S. Application Data
(63) Continuation of application No. PCT/KR2025/006793, filed on May 19, 2025.

(30) Foreign Application Priority Data
May 21, 2024 (KR) 10-2024-0066112

(51) Int. Cl. A61B 6/50; A61B 6/42

(52) U.S. Cl. CPC A61B 6/502; A61B 6/4208

(57) ABSTRACT

A mammography apparatus includes a radiation source irradiating radiation onto a breast of a patient and a radiation detector disposed facing the radiation source, the radiation detector generating a radiation image of first energy and a radiation image of second energy based on radiation that has passed through the breast of the patient. The radiation detector includes a first radiation detection unit comprising a first detection panel having a first surface and a second surface parallel to each other and a first light conversion layer provided on the first surface of the first detection panel, the first radiation detection unit generating the radiation image of the first energy, and a second radiation detection unit comprising a second detection panel having a first surface and a second surface parallel to each other and a second light conversion layer provided on the first surface of the second detection panel.

### CROSS-REFERENCE TO RELATED APPLICATIONS

This application is a continuation of International Application No. PCT/KR2025/006793 filed on May 19, 2025, which claims priority to Korean Patent Application No. 10-2024-0066112 filed on May 21, 2024, the entire contents of which are herein incorporated by reference.

### TECHNICAL FIELD

The present disclosure relates to a radiation detector and a radiographic apparatus including the same, and more specifically, relates to a radiation detector capable of acquiring radiation images for radiation of a plurality of energies and a radiographic apparatus including the same. The radiographic apparatus may include a mammography apparatus.

### BACKGROUND ART

Radiation such as X-rays accompanies a constant attenuation phenomenon according to the energy of radiation and the type of material, such as Compton scattering and photoelectric effect, during the process of passing through an object.

Radiography is a radiation imaging method that uses such an attenuation phenomenon to project and display the internal structure of a subject on a plane, wherein radiation is generated from a radiation source and irradiated onto a subject, the radiation that has passed through the subject is detected by a radiation detector, and the intensity of radiation at each position is displayed as a radiation image such as a gray scale.

Assuming that the subject is a patient, the intensity of radiation detected by the radiation detector is affected by the radiation attenuation coefficient according to the radiation energy and the characteristics of constituent materials of body tissue located on the path of radiation (such as atomic number and density). For example, since the densities of air, fat, water, and bone in the human body are in the order of 0.00129, 0.91, 1, and 1.85 g/cm while the average atomic number also increases in the same order, the attenuation coefficient of radiation increases in the order of air, fat, water, and bone, and the intensity of radiation detected by the radiation detector decreases in the corresponding order.

Meanwhile, due to the radiation energy dependence of such an attenuation phenomenon, the energy level of radiation for obtaining a desired diagnostic image may differ depending on the body tissue.

For example, radiation of a relatively low energy level is suitable for a diagnostic image of soft tissue such as skin, and radiation of a relatively high energy level is suitable for a diagnostic image of hard tissue such as bone or teeth.

However, since soft tissue and hard tissue are complexly distributed in the human body, there is a clear limit to the quality of a diagnostic image obtained with radiation of a certain energy level. That is, it is difficult to obtain a high-quality diagnostic image emphasizing a specific body tissue (for example, a lesion) with radiation of a certain energy level.

The conventional dual energy technique utilizes the fact that the attenuation coefficient of a material varies depending on the incident X-ray energy, and this technique performs two X-ray irradiations to acquire low-and high-energy images, wherein the two X-ray irradiations increase the radiation dose, and since complete separation of the X-ray spectrum is impossible, the accuracy of material separation is reduced and image quality is degraded.

In addition, since a time difference exists between radiographic captures, motion artifacts occur due to movement of organs or a subject (for example, heartbeat, breathing, etc.), and not only the imaging time and imaging cost increase, but also the radiation dose to the subject may increase.

Therefore, a dual energy radiographic technique capable of improving the quality of radiation images while reducing the radiation dose to a subject is required.

### PRIOR ART DOCUMENT

### Patent Document

(Patent Document 1) Korean Patent Publication No. 10-2016-0079961.

### SUMMARY

### Technical Problem

The present disclosure provides a radiation detector capable of acquiring a radiation image with excellent image uniformity for radiation of first and second energies, and a radiographic apparatus including the same.

### Technical Solution

A radiation detector according to one embodiment of the present disclosure includes a first radiation detection unit comprising a first detection panel having a first surface and a second surface parallel to each other, and a first light conversion layer provided on the first surface of the first detection panel; and a second radiation detection unit comprising a second detection panel having a first surface and a second surface parallel to each other, and a second light conversion layer provided on the first surface of the second detection panel, wherein the first radiation detection unit and the second radiation detection unit may be disposed such that the second surface of the first detection panel and the second surface of the second detection panel face each other.

The first radiation detection unit may detect radiation of first energy from radiation incident from a first surface side of the first detection panel, and the second radiation detection unit may detect radiation of second energy from radiation that has passed through the first radiation detection unit and is incident from a second surface side of the second detection panel.

The first energy and the second energy may be different.

The first light conversion layer and the second light conversion layer may be made of different materials.

The radiation detector may further include a light blocking layer provided between the first light conversion layer and the second light conversion layer, the light blocking layer blocking light converted by the first light conversion layer from being incident onto the second detection panel or light converted by the second light conversion layer from being incident onto the first detection panel.

The first light conversion layer and the second light conversion layer may have different thicknesses from each other.

The first detection panel and the second detection panel may each include a pixel array composed of a plurality of pixels, and the pixel array of the first detection panel and the pixel array of the second detection panel may have different sizes of the plurality of pixels.

At least one of the first light conversion layer and the second light conversion layer may include a scintillator material converting radiation into visible light.

The first light conversion layer and the second light conversion layer may be made of cesium iodide (CsI).

The first light conversion layer and the second light conversion layer may be made of gadolinium oxysulfide (GOS).

At least one of the first light conversion layer and the second light conversion layer may include a photoelectric material generating an electrical signal by radiation.

Among the first light conversion layer and the second light conversion layer, a light conversion layer into which radiation is first incident may include a photoelectric material generating an electrical signal by the radiation, and among the first light conversion layer and the second light conversion layer, a light conversion layer into which the radiation is subsequently incident may include a scintillator material converting the radiation into visible light.

The first light conversion layer may be made of selenium (Se), and the second light conversion layer may be made of gadolinium oxysulfide (GOS).

The first light conversion layer may be made of selenium (Se), and the second light conversion layer may be made of cesium iodide (CsI).

At least one of the first detection panel and the second detection panel may be flexible.

The radiation detector may further include a support member supporting at least one of the first detection panel and the second detection panel.

The radiation detector may further include a third light conversion layer provided between the first detection panel and the second detection panel.

The third light conversion layer may be identical to at least one of the first light conversion layer and the second light conversion layer.

A radiographic apparatus according to another embodiment of the present disclosure may include a radiation detector according to one embodiment of the present disclosure; a radiation source irradiating radiation toward the radiation detector; and an image processing unit processing the radiation of the first energy detected by the first radiation detection unit and the radiation of the second energy detected by the second radiation detection unit to generate a radiation image.

A mammography apparatus according to one embodiment of the present disclosure includes a radiation source irradiating radiation onto a breast of a patient and a radiation detector disposed facing the radiation source, the radiation detector generating a radiation image of first energy and a radiation image of second energy based on radiation that has passed through the breast of the patient, wherein the radiation detector includes a first radiation detection unit comprising a first detection panel having a first surface and a second surface parallel to each other and a first light conversion layer provided on the first surface of the first detection panel, the first radiation detection unit generating the radiation image of the first energy, and a second radiation detection unit comprising a second detection panel having a first surface and a second surface parallel to each other and a second light conversion layer provided on the first surface of the second detection panel, the second radiation detection unit generating the radiation image of the second energy, wherein the first radiation detection unit and the second radiation detection unit are disposed such that the second surface of the first detection panel and the second surface of the second detection panel face each other.

The first radiation detection unit of the mammography apparatus according to one embodiment of the present disclosure detects radiation of first energy from radiation incident from a first surface side of the first detection panel, the second radiation detection unit detects radiation of second energy from radiation that has passed through the first radiation detection unit and is incident from a second surface side of the second detection panel, and the first energy and the second energy are different.

At least one of the first light conversion layer and the second light conversion layer of the mammography apparatus according to one embodiment of the present disclosure includes a scintillator material converting radiation into visible light.

The first light conversion layer and the second light conversion layer of the mammography apparatus according to one embodiment of the present disclosure are made of cesium iodide (CsI).

The first light conversion layer and the second light conversion layer of the mammography apparatus according to one embodiment of the present disclosure are made of gadolinium oxysulfide (GOS).

At least one of the first light conversion layer and the second light conversion layer of the mammography apparatus according to one embodiment of the present disclosure includes a photoelectric material generating an electrical signal by radiation.

Among the first light conversion layer and the second light conversion layer of the mammography apparatus according to one embodiment of the present disclosure, a light conversion layer into which radiation is first incident includes a photoelectric material generating an electrical signal by the radiation, and among the first light conversion layer and the second light conversion layer, a light conversion layer into which the radiation is subsequently incident includes a scintillator material converting the radiation into visible light.

The first light conversion layer of the mammography apparatus according to one embodiment of the present disclosure is made of selenium (Se), and the second light conversion layer is made of gadolinium oxysulfide (GOS).

The first light conversion layer of the mammography apparatus according to one embodiment of the present disclosure is made of selenium (Se), and the second light conversion layer is made of cesium iodide (CsI).

The mammography apparatus according to one embodiment of the present disclosure includes a controller for controlling the mammography apparatus, wherein the controller acquires the radiation image of the first energy using the first radiation detection unit, the controller acquires the radiation image of the second energy using the second radiation detection unit, and the controller acquires a synthesized image based on the radiation image of the first energy and the radiation image of the second energy.

The controller of the mammography apparatus according to one embodiment of the present disclosure acquires a first corrected image by applying the radiation image of the first energy to an image enhancement model in order to acquire the synthesized image, acquires a second corrected image by applying the radiation image of the second energy to the image enhancement model, and acquires a synthesized image based on the first corrected image and the second corrected image.

The controller of the mammography apparatus according to one embodiment of the present disclosure acquires noise information by applying at least one of the radiation image of the first energy or the radiation image of the second energy to a noise model, and acquires an improved synthesized image from which noise is removed from the synthesized image based on the noise information.

The radiation image of the first energy of the mammography apparatus according to one embodiment of the present disclosure is a radiation image of lower energy compared to the radiation image of the second energy.

### Advantageous Effects

In a radiation detector according to an embodiment of the present disclosure, by disposing the first radiation detection unit and the second radiation detection unit such that the second surface of the first detection panel and the second surface of the second detection panel face each other, the first detection panel and the second detection panel may have excellent thickness uniformity such that the distance between the first light conversion layer and the second light conversion layer may be generally constant, and accordingly, the image uniformity between the radiation image of the first energy generated through the first radiation detection unit and the radiation image of the second energy generated through the second radiation detection unit may be improved.

At this time, by providing a light blocking layer between the first light conversion layer and the second light conversion layer, it is possible to prevent light converted by the first light conversion layer from affecting the second detection panel or light converted by the second light conversion layer from affecting the first detection panel.

In addition, by forming at least one of the first detection panel and the second detection panel as a flexible panel to make it thin, the distance between the first light conversion layer and the second light conversion layer may be reduced, and accordingly, the reduction of radiation by the first detection panel and/or the second detection panel may be reduced, and parallax effects such as magnification differences between the radiation image of the first energy generated through the first radiation detection unit and the radiation image of the second energy generated through the second radiation detection unit may also be reduced.

### DESCRIPTION OF DRAWINGS

FIG. 1 is a schematic cross-sectional view illustrating a radiation detector according to one embodiment of the present disclosure.

FIG. 2 is a schematic cross-sectional view illustrating a radiation detector including a light blocking layer according to one embodiment of the present disclosure.

FIG. 3 is a schematic cross-sectional view illustrating a radiation detector including first and second light conversion layers having different thicknesses according to one embodiment of the present disclosure.

FIGS. 4A, 4B, 4C are schematic cross-sectional views illustrating modifications of a radiation detector according to embodiments of the present disclosure.

FIG. 5 is a schematic cross-sectional view illustrating a radiographic apparatus according to another embodiment of the present disclosure.

FIG. 6 illustrates a mammography apparatus according to one embodiment of the present disclosure.

FIG. 7 is a view for describing an operation of the mammography apparatus according to one embodiment of the present disclosure.

FIG. 8 is a view for describing an operation of the mammography apparatus according to one embodiment of the present disclosure.

### DETAILED DESCRIPTION

Hereinafter, embodiments of the present disclosure will be described in more detail with reference to the accompanying drawings. However, the present disclosure is not limited to the embodiments disclosed below but will be implemented in various different forms, and the embodiments are provided only to make the disclosure of the present disclosure complete and to fully inform those of ordinary skill in the art of the scope of the disclosure. In the description, the same reference numerals are assigned to the same components, and the drawings may be partially exaggerated in size to accurately describe the embodiments of the present disclosure, and the same reference numerals in the drawings refer to the same elements.

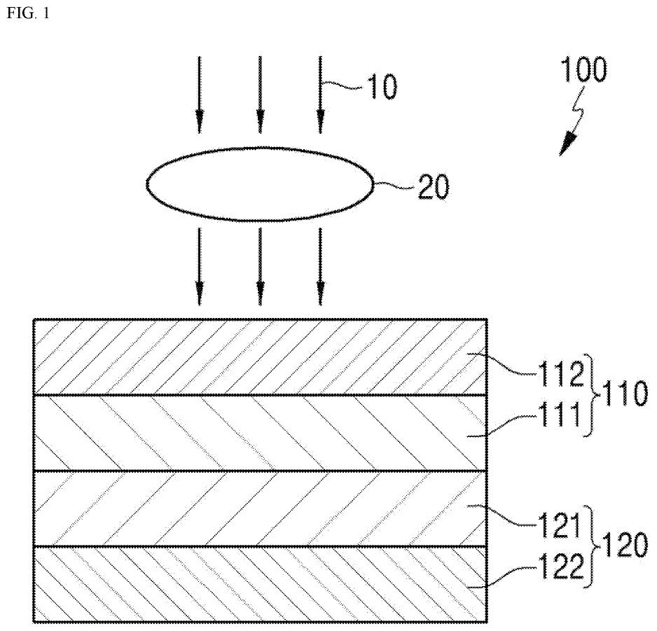

FIG. 1 is a schematic cross-sectional view illustrating a radiation detector according to one embodiment of the present disclosure.

Referring to FIG. 1, a radiation detector 100 according to one embodiment of the present disclosure may include a first radiation detection unit 110 comprising a first detection panel 111 having a first surface and a second surface parallel to each other, and a first light conversion layer 112 provided on the first surface of the first detection panel 111; and a second radiation detection unit 120 comprising a second detection panel 121 having a first surface and a second surface parallel to each other, and a second light conversion layer 122 provided on the first surface of the second detection panel 121.

The first radiation detection unit 110 may detect radiation 10 such as X-rays, and may convert image information of the radiation 10 into an electrical signal for detection. For example, the first radiation detection unit 110 may have a first surface and a second surface facing each other, and among the first surface and the second surface, a surface facing the radiation source 210 may be a radiation transmission surface, and the radiation 10 incident onto the radiation transmission surface may be converted into an electrical signal capable of image signal processing. At this time, the first radiation detection unit 110 may detect radiation of first energy (or energy band).

Here, the first radiation detection unit 110 may include a first detection panel 111 having a first surface and a second surface parallel to each other, and a first light conversion layer 112 provided on the first surface of the first detection panel 111. The first detection panel 111 may have a first surface and a second surface parallel to (or facing) each other, and the first surface and the second surface may have excellent flatness and may have a uniform thickness overall. For example, the first detection panel 111 may include a pixel circuit board, the pixel circuit board may include a thin film transistor (TFT), and in the case of an indirect method, the first detection panel 111 may (further) include a light-receiving element such as a photodiode that senses light converted by the first light conversion layer 112 such as a scintillator and converts the light into an electrical signal, and the light-receiving element may be an amorphous silicon (α-Si) photodiode circuit.

The first light conversion layer 112 may be provided on the first surface of the first detection panel 111, and may be a photoconductive layer (direct method) composed of selenium (Se) such as amorphous selenium (α-Se), cadmium telluride (CdTe) such as crystalloid CdTe, a crystalloid cadmium telluride compound, or the like, or may be a scintillator (layer) (indirect method) composed of cesium iodide (CsI), thallium (Tl)-doped cesium iodide (CsI(Tl)), gadolinium oxysulfide (Gadolinium Oxysulfide; GOS, GdxOySz or Gadox, Gd2O2S), calcium tungstate (CaWOx), or the like. In the case of a direct method of directly detecting an electrical signal generated by radiation 10 that has passed through a subject 20, the photoconductive layer exhibiting photoconductive properties and directly converting radiation 10 into an electrical signal through a photoelectric effect may be used as the first light conversion layer 112, and in the case of the indirect method of converting radiation 10 into light such as visible light in a fluorescent layer such as a scintillator and converting the converted light back into an electrical signal, the scintillator (layer) converting radiation 10 into optical photons (i.e., light) may be used as the first light conversion layer 112.

The second radiation detection unit 120 may also detect radiation 10, and may convert image information of the radiation 10 into an electrical signal for detection. For example, the second radiation detection unit 120 may have a first surface and a second surface facing each other, and among the first surface and the second surface, a surface facing the radiation source 210 may be a radiation transmission surface, and the radiation 10 incident onto the radiation transmission surface may be converted into an electrical signal capable of image signal processing. At this time, the second radiation detection unit 120 may detect radiation of second energy (or energy band), and may also detect radiation that has passed through the first radiation detection unit 110.

Here, the second radiation detection unit 120 may include a second detection panel 121 having a first surface and a second surface parallel to each other, and a second light conversion layer 122 provided on the first surface of the second detection panel 121. The second detection panel 121 may have a first surface and a second surface parallel to each other, and the first surface and the second surface may have excellent flatness and may have a uniform thickness overall. For example, the second detection panel 121 may also include a pixel circuit board, the pixel circuit board may include a thin film transistor (TFT), and in the case of the indirect method, similar to the first detection panel 111, the second detection panel 121 may (further) include a light-receiving element such as a photodiode that senses light converted by the second light conversion layer 122 such as a scintillator and converts the light into an electrical signal, and the light-receiving element may be an amorphous silicon (α-Si) photodiode circuit.

The second light conversion layer 122 may be provided on the first surface of the second detection panel 121, and similar to the first light conversion layer 112, depending on the radiation detection method selected from the direct method and the indirect method, the second light conversion layer 122 may be a photoconductive layer (direct method) composed of selenium (Se) such as amorphous selenium (α-Se), cadmium telluride (CdTe) such as crystalloid CdTe, a crystalloid cadmium telluride compound, or the like, or a scintillator (layer) (indirect method) composed of cesium iodide (CsI), thallium (Tl)-doped cesium iodide (CsI(Tl)), gadolinium oxysulfide (GOS or Gadox), calcium tungstate (CaWOx), or the like.

In addition, the first light conversion layer 112 and the second light conversion layer 122 may not be flat, may be less flat than the first detection panel 111 and the second detection panel 121, and may not have a uniform thickness overall, such that the thickness may differ for each region (or position). For example, the scintillator (layer) made of cesium iodide (CsI), which is mainly used as the first light conversion layer 112 and/or the second light conversion layer 122, is composed of columnar crystals and may not be flat due to the cesium iodide (CsI) deposition process method.

Here, the first radiation detection unit 110 and the second radiation detection unit 120 may be disposed such that the second surface of the first detection panel 111 and the second surface of the second detection panel 121 face each other, the second surface of the first detection panel 111 and the second surface of the second detection panel 121 may (directly) contact each other, and other components such as a light blocking layer 131 may be interposed between the second surface of the first detection panel 111 and the second surface of the second detection panel 121. That is, the first radiation detection unit 110 and the second radiation detection unit 120 may be symmetrically disposed such that the second surface of the first detection panel 111 and the second surface of the second detection panel 121 face each other, and the first light conversion layer 112 and the first detection panel 111 may be symmetric with the second detection panel 121 and the second light conversion layer 122 with respect to a center between the second surface of the first detection panel 111 and the second surface of the second detection panel 121. Through this, the radiation detector 100 in which radiation 10 is incident onto the first radiation detection unit 110 from the first surface side of the first detection panel 111 (front incidence) and radiation 10 is incident onto the second radiation detection unit 120 from the second surface side of the second detection panel 121 (back incidence) may be formed.

Since the first detection panel 111 and the second detection panel 121 have excellent thickness uniformity, when the first radiation detection unit 110 and the second radiation detection unit 120 are symmetrically disposed such that the second surface of the first detection panel 111 and the second surface of the second detection panel 121 face each other, the distance between the first light conversion layer 112 and the second light conversion layer 122 (and/or the distance between the first radiation detection unit and the second radiation detection unit) may be generally (or overall) constant, and accordingly, the image uniformity between the radiation image of the first energy generated through the first radiation detection unit 110 and the radiation image of the second energy generated through the second radiation detection unit 120 may be improved.

In addition, for image uniformity between the radiation image of the first energy and the radiation image of the second energy, alignment between pixels of the first detection panel 111 and the second detection panel 121 is important, and when the first radiation detection unit 110 and the second radiation detection unit 120 are symmetrically disposed such that the second surface of the first detection panel 111 and the second surface of the second detection panel 121 face each other, the distance between the first detection panel 111 and the second detection panel 121 may also become closer, making it easy not only to align the centers of the pixels of the first detection panel 111 and the second detection panel 121, but also the distance between the pixels of the first detection panel 111 and the second detection panel 121 may be overall constant, and the pixels of the first detection panel 111 and the pixels of the second detection panel 121 may also be parallel and overlap each other. Through this, the image uniformity between the radiation image of the first energy and the radiation image of the second energy may be further improved.

Meanwhile, when the first detection panel 111 and/or the second detection panel 121 includes a transparent material, alignment between pixels of the first detection panel 111 and the second detection panel 121 may be facilitated, and the pixels of the first detection panel 111 and the second detection panel 121 may be aligned while actually checking (or viewing) the pixel positions of the first detection panel 111 and the second detection panel 121. At this time, the pixel positions of the first detection panel 111 and the second detection panel 121 may be measured and confirmed using an optical sensor such as a camera.

The first radiation detection unit 110 and the second radiation detection unit 120 may be arranged (or stacked) in the irradiation direction of radiation 10, the first radiation detection unit 110 may detect radiation of first energy from radiation 10 incident from the first surface side of the first detection panel 111, and the second radiation detection unit 120 may detect radiation of second energy from radiation incident from the second surface side of the second detection panel 121 after passing through the first radiation detection unit 110.

For example, the radiation source 210 may be provided on the first surface side of the first detection panel 111 and may irradiate radiation 10 toward the first radiation detection unit 110, the radiation 10 may be incident onto the first light conversion layer 112 and at least a portion thereof may be converted, and among the remaining portion of the radiation 10, the radiation that has passed through (or transmitted through) the first radiation detection unit 110 may pass through the second detection panel 121, reach the second light conversion layer 122, and be converted. That is, the radiation 10 irradiated from the radiation source 210 may be incident in the order (or sequence) of the first light conversion layer 112, the first detection panel 111, the second detection panel 121, and the second light conversion layer 122.

When the first radiation detection unit 110 and the second radiation detection unit 120 are arranged in the irradiation direction of the radiation 10, the distances of the first radiation detection unit 110 and the second radiation detection unit 120 from the radiation source 210 become different, and as a result, a magnification difference occurs between the radiation image generated by the first radiation detection unit 110 and the radiation image generated by the second radiation detection unit 120. At this time, when the distance between the first radiation detection unit 110 and the second radiation detection unit 120 (for example, the distance between the first light conversion layer and the second light conversion layer) varies for each position (or region), a deviation in the magnification difference occurs at each position.

The scintillator (layer) of the direct deposition method mainly used as the first light conversion layer 112 and/or the second light conversion layer 122 has (relatively) a thickness deviation at each position, which may result in different magnifications (or magnification deviations) at different positions, and as a result, a deviation in the magnification difference occurs between the radiation image generated by the first radiation detection unit 110 and the radiation image generated by the second radiation detection unit 120, and errors may easily occur during image registration work for matching two images having the deviation in the magnification difference, which may cause image artifacts. When the deviation in the magnification difference causes poor image registration between the two images and image artifacts occur, the performance of a (diagnostic) image obtained by subtraction and/or synthesis of the two images is reduced.

However, since the first detection panel 111 and the second detection panel 121 have a (relatively) very uniform thickness, when the first radiation detection unit 110 and the second radiation detection unit 120 are disposed back-to-back such that the second surface of the first detection panel 111 and the second surface of the second detection panel 121 face each other, the distance between the first radiation detection unit 110 and the second radiation detection unit 120 (for example, the distance between the first light conversion layer and the second light conversion layer) may be constant throughout the entire detection area (or overall), and accordingly, the deviation in the magnification difference may not occur between the radiation image generated by the first radiation detection unit 110 and the radiation image generated by the second radiation detection unit 120, and the image uniformity between the two radiation images may be improved. Accordingly, image registration between the radiation image generated by the first radiation detection unit 110 and the radiation image generated by the second radiation detection unit 120 may be facilitated, and a clear subtraction image and/or synthesized image may be acquired using the two radiation images without degradation of image quality, and the quality of the (diagnostic) image may be improved, thereby improving the accuracy of image diagnosis.

Meanwhile, since the first detection panel 111 and the second detection panel 121 have a first surface with excellent flatness, when the first light conversion layer 112 and the second light conversion layer 122 are deposited on the first surface of the first detection panel 111 and the first surface of the second detection panel 121, respectively, the deposition thicknesses of the first light conversion layer 112 and the second light conversion layer 122 may be formed uniformly, and may also be controlled to a desired deposition thickness. For example, the first detection panel 111 and the second detection panel 121 may have a constant size such as thickness and area, and based on information on the thickness, area, and the like of the constant first detection panel 111 and second detection panel 121, the deposition thickness, area, position, and the like of the first light conversion layer 112 and the second light conversion layer 122 may be completely changed (shifted) and controlled according to a desired (or required) specification. Accordingly, the accurate deposition thicknesses of the first light conversion layer 112 and the second light conversion layer 122 according to the specification may be uniformly formed and manufactured, and image quality may be increased during image registration of the radiation image generated by the first radiation detection unit 110 and the radiation image generated by the second radiation detection unit 120.

The first energy and the second energy may be different. That is, the radiation detector 100 of the present disclosure may be a dual energy radiation detector, and may acquire radiation images of a plurality of energies (for example, dual energy) (bands), and for this purpose, the first radiation detection unit 110 and the second radiation detection unit 120 may detect radiation of different (or mutually different) energies (or energy bands). At this time, the first radiation detection unit 110 may detect radiation of the first energy, and the second radiation detection unit 120 may detect radiation of the second energy different from the first energy. For example, the first radiation detection unit 110 and the second radiation detection unit 120 may completely overlap to acquire a dual energy radiation image, and a dual energy radiation image may be acquired corresponding to the area of the radiation transmission surfaces of the first radiation detection unit 110 and the second radiation detection unit 120.

At this time, the first detection panel 111 and the second detection panel 121 may be formed of a radiation-transmitting material, and radiation (for example, high-energy radiation) that has been incident onto the first radiation detection unit 110 and passed through (or transmitted through) the first detection panel 111 may be transmitted through the second detection panel 121 and delivered to the second light conversion layer 122, and the radiation thus delivered may be converted in the second light conversion layer 122. Through this, by detecting radiation of different energies by the first radiation detection unit 110 and the second radiation detection unit 120, respectively, a dual energy radiation image may be acquired.

The first radiation detection unit 110 and the second radiation detection unit 120 may detect radiation of different energies in an overlapping region, may acquire a dual energy radiation image for the overlapping region, and may perform dual energy imaging with a single irradiation of radiation 10.

Here, when the first radiation detection unit 110 is located closer to the radiation source 210 than the second radiation detection unit 120, the first radiation detection unit 110 may detect and filter (or remove) low-energy radiation, thereby delivering only high-energy radiation to the second radiation detection unit 120, and the second radiation detection unit 120 may detect high-energy radiation delivered by passing through the first radiation detection unit 110.

At this time, the first light conversion layer 112 and the second light conversion layer 122 may be different. In order for an energy difference (for example, an image difference between bone and soft tissue) to clearly appear between the radiation image of the first energy (for example, low energy) acquired by the first radiation detection unit 110 and the radiation image of the second energy (for example, high energy) acquired by the second radiation detection unit 120, the first radiation detection unit 110 must effectively detect (only) radiation of the first energy, and the second radiation detection unit 120 must effectively detect (only) radiation of the second energy.

The energy of radiation absorbed and/or converted by the first light conversion layer 112 and the second light conversion layer 122 may differ depending on the material and/or thickness of the first light conversion layer 112 and the second light conversion layer 122, and accordingly, the radiation energy detectable by the first radiation detection unit 110 and the second radiation detection unit 120 may differ. Accordingly, the first light conversion layer 112 may be formed of a material and/or thickness that absorbs and/or converts the first energy well, and the second light conversion layer 122 may be formed of a material and/or thickness that absorbs and/or converts the second energy well, and since the first radiation detection unit 110 and the second radiation detection unit 120 detect radiation of the mutually different first energy and second energy, respectively, the first light conversion layer 112 and the second light conversion layer 122 may be different.

For example, the first light conversion layer 112 and the second light conversion layer 122 may be made of different materials, and the energy characteristics of detectable radiation may differ depending on the materials of the first light conversion layer 112 and the second light conversion layer 122. When the first radiation detection unit 110 detects low-energy radiation, the first light conversion layer 112 may be formed of cesium iodide (CsI) having high sensitivity to low-energy radiation, and when the second radiation detection unit 120 detects high-energy radiation, the second light conversion layer 122 may be formed of gadolinium oxysulfide (GOS) having high sensitivity to high-energy radiation.

Meanwhile, the materials of the first light conversion layer 112 and the second light conversion layer 122 may be different when the first radiation detection unit 110 is configured for the direct method and the second radiation detection unit 120 is configured for the indirect method.

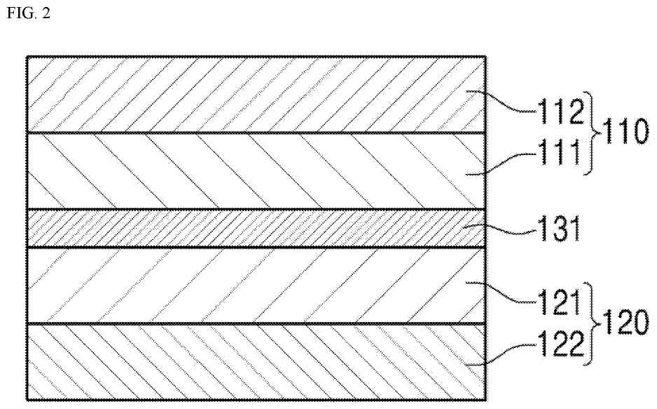

FIG. 2 is a schematic cross-sectional view illustrating a radiation detector including a light blocking layer according to one embodiment of the present disclosure.

Referring to FIG. 2, the radiation detector 100 according to the present disclosure may further include a light blocking layer 131 provided between the first light conversion layer 112 and the second light conversion layer 122, the light blocking layer 131 blocking light converted by the first light conversion layer 112 from being incident onto the second detection panel 121, or blocking light converted by the second light conversion layer 122 from being incident onto the first detection panel 111.

The light blocking layer 131 may be provided between the first light conversion layer 112 and the second light conversion layer 122, and may block light (for example, visible light) converted (or generated) by the first light conversion layer 112 from being incident onto the second detection panel 121, or block light converted by the second light conversion layer 122 from being incident onto the first detection panel 111. For example, the light blocking layer 131 may be interposed between the first detection panel 111 and the second detection panel 121, may block light converted by the first light conversion layer 112 from being incident onto the second detection panel 121, and may block light converted by the second light conversion layer 122 from being incident onto the first detection panel 111. Accordingly, light converted by the first light conversion layer 112 may be incident (only) onto the first detection panel 111 and not incident onto the second detection panel 121, and light converted by the second light conversion layer 122 may be incident (only) onto the second detection panel 121 and not incident onto the first detection panel 111. Here, the light blocking layer 131 may be radiation-transmissive, may block visible light, and may transmit at least a portion of radiation to deliver radiation 10 between the first radiation detection unit 110 and the second radiation detection unit 120.

The first detection panel 111 and the second detection panel 121 may be made of a transparent material so that loss of radiation 10 does not occur in the first detection panel 111 and/or the second detection panel 121, and as a result, light converted by the first light conversion layer 112 and the second light conversion layer 122 may affect the detection panels 121 and 111 of other radiation detection units 120 and 110 (for example, a detection panel on which no deposition has been performed) rather than the detection panels 111 and 121 of the corresponding (or included) radiation detection units 110 and 120. Accordingly, the light blocking layer 131 may be formed between the first detection panel 111 and the second detection panel 121 to prevent light converted by the first light conversion layer 112 and the second light conversion layer 122 from affecting the detection panels 121 and 111 of the other radiation detection units 120 and 110.

That is, the light blocking layer 131 may be disposed between the first detection panel 111 and the second detection panel 121 to prevent crossover or interference of light converted by the first light conversion layer 112 and the second light conversion layer 122 with respect to the detection panels 121 and 111 of the other radiation detection units 120 and 110, may block light delivered from the light conversion layers 122 and 112 of the other radiation detection units 120 and 110 between the first detection panel 111 and the second detection panel 121, and may have the effect of eliminating signal interference between the first detection panel 111 and the second detection panel 121. At this time, the light blocking layer 131 may be a light-absorbing blocking layer that absorbs and blocks visible light.

For example, the light blocking layer 131 may be composed of an organic photoelectric conversion film made of an organic photoelectric conversion material, and may be made of Poron or high-density (poly)urethane, or the like. In addition, the light blocking layer 131 may also be a hydrophilic colloid layer of gelatin containing a mixture of microcrystalline crossover-reducing dyes such as arylidene dyes in a 1:1 weight ratio.

Meanwhile, the light blocking layer 131 may also serve as a radiation (for example, X-ray) energy filter, and may absorb and remove at least some energy (for example, the first energy) from radiation 10. For example, the light blocking layer 131 may absorb and remove low-energy (or the first energy) radiation, thereby delivering (or providing) only high-energy (or the second energy) radiation to the second radiation detection unit 120. The light blocking layer 131 may include a metal or alloy as a radiation energy filter, and may include any one or a combination of two or more of copper (Cu), aluminum (Al), silver (Ag), titanium (Ti), vanadium (V), chromium (Cr), iron (Fe), cobalt (Co), nickel (Ni), zinc (Zn), zirconium (Zr), niobium (Nb), molybdenum (Mo), cadmium (Cd), tin (Sn), barium (Ba), tungsten (W), tantalum (Ta), gold (Au), and lead (Pb). In addition, the light blocking layer 131 may also perform noise blocking between the first detection panel 111 and the second detection panel 121.

Therefore, the radiation detector 100 according to the present disclosure may provide the light blocking layer 131 between the first light conversion layer 112 and the second light conversion layer 122 to prevent light converted by the first light conversion layer 112 from affecting the second detection panel 121 or light converted by the second light conversion layer 122 from affecting the first detection panel 111, and accordingly, may have the effect of eliminating signal interference between the first detection panel 111 and the second detection panel 121, and may improve the image quality of radiation images respectively generated by the first radiation detection unit 110 and the second radiation detection unit 120.

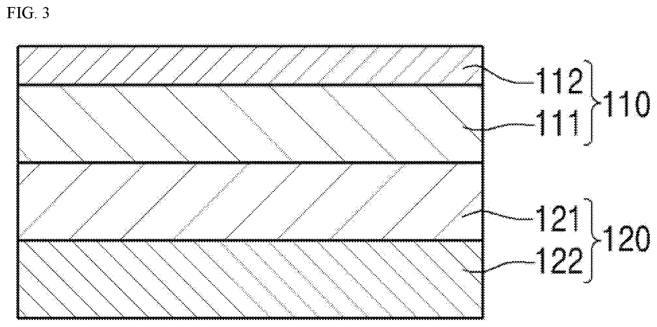

FIG. 3 is a schematic cross-sectional view illustrating a radiation detector including first and second light conversion layers having different thicknesses according to one embodiment of the present disclosure.

Referring to FIG. 3, the first light conversion layer 112 and the second light conversion layer 122 may have different thicknesses from each other, and the energy (or energy band) of detectable radiation may differ depending on the thicknesses of the first light conversion layer 112 and the second light conversion layer 122. For example, when the first radiation detection unit 110 detects low-energy radiation, the first light conversion layer 112 may be formed (relatively) thin so as to detect low-energy radiation well, and when the second radiation detection unit 120 detects high-energy radiation, the second light conversion layer 122 may be formed (relatively) thick so as to detect high-energy radiation well.

In addition, since the amount of light emission (i.e., the amount of radiation converted into light in the first light conversion layer) increases as the first light conversion layer 112 becomes thicker, making the first light conversion layer 112, which has a (relatively) high absorption rate due to almost no (or few) obstacles (for example, radiation absorbers) between the radiation source 210 and itself, thinner than the second light conversion layer 122 may be better in terms of image quality and sensitivity.

In addition, the first detection panel 111 and the second detection panel 121 may each include a pixel array composed of a plurality of pixels, and the pixel array of the first detection panel 111 and the pixel array of the second detection panel 121 may differ in at least one of the size and spacing of the plurality of pixels. Each of the first detection panel 111 and the second detection panel 121 may include a pixel array composed of a plurality of pixels, and may be a flat panel in which the plurality of pixels are arranged in a matrix form along a two-dimensional plane.

Here, the pixel array of the first detection panel 111 and the pixel array of the second detection panel 121 may differ in at least one of the size and spacing of the plurality of pixels, and the size and/or spacing of the plurality of pixels of the second detection panel 121 may be adjusted (or tuned) according to the magnification difference due to the difference in distance from the radiation source 210 between the first detection panel 111 and the second detection panel 121, and the size and/or spacing of the plurality of pixels may be made different between the pixel array of the first detection panel 111 and the pixel array of the second detection panel 121. In addition, each of the first radiation detection unit 110 and the second radiation detection unit 120 may be configured as sensitivity-centered or image quality-centered according to the size of the plurality of pixels, and when the size of each pixel increases and the light-receiving area increases, sensitivity may be improved to become sensitivity-centered, and when the size of each pixel decreases and the light-receiving area decreases, image quality may be improved to become image quality-centered. For example, the first radiation detection unit 110 having the first light conversion layer 112 with a (relatively) high absorption rate may be configured as image quality-centered, and the second radiation detection unit 120 having the second light conversion layer 122, which has a (relatively) low absorption rate due to (relatively) many obstacles between the radiation source 210 and itself, may be configured as sensitivity-centered. At this time, the size of the plurality of pixels in the pixel array of the first detection panel 111 may be (relatively) smaller than the plurality of pixels of the pixel array of the second detection panel 121 so that the first radiation detection unit 110 becomes image quality-centered, and the size of the plurality of pixels in the pixel array of the second detection panel 121 may be (relatively) larger than the plurality of pixels of the pixel array of the first detection panel 111 so that the second radiation detection unit 120 becomes sensitivity-centered.

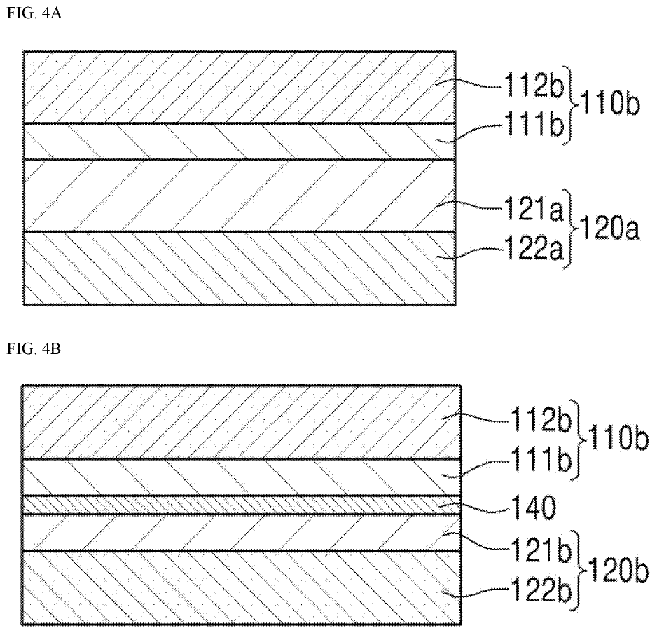

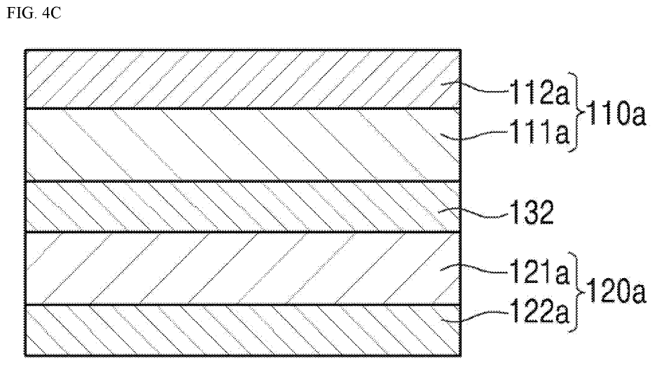

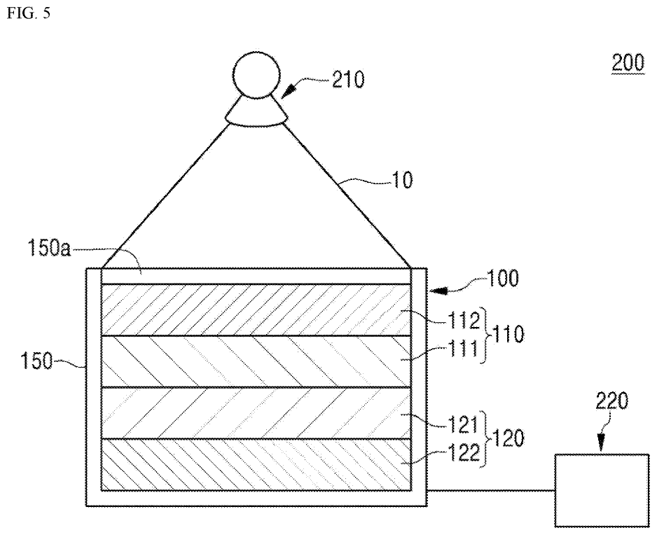

FIGS. 4A, 4B, and 4C are schematic cross-sectional views illustrating modifications of a radiation detector according to embodiments of the present disclosure, wherein FIG. 4A illustrates a radiation detector composed of a direct-method first radiation detection unit and an indirect-method second radiation detection unit, FIG. 4B illustrates a radiation detector in which a support member is provided between a direct-method first radiation detection unit and a direct-method second radiation detection unit, and FIG. 4C illustrates a radiation detector in which a third light conversion layer is provided between an indirect-method first radiation detection unit and an indirect-method second radiation detection unit.

Referring to FIGS. 4A, 4B, and 4C, at least one of the first light conversion layer 112 and the second light conversion layer 122 may include a scintillator material converting radiation 10 into visible light, and a radiation detection unit 110a or 120a having a light conversion layer 112a or 122a including a scintillator material converting radiation 10 into visible light among the first light conversion layer 112 and the second light conversion layer 122 may be the indirect method of converting visible light, into which radiation 10 has been converted by the scintillator material, back into an electrical signal. Here, the scintillator material may include rare earth oxysulfides doped with a rare earth activator, and may be any one or a combination of Gd2O2S:Tb, Gd2O2S:Eu, Gd2O3:Eu, La2O2S:Tb, La2O2S, Y2O2S:Tb, CsI:Tl, CsI:Na, CsBr:Tl, NaI:Tl, CaWO4, CaWO4:Tb, BaFBr:Eu, BaFCl:Eu, BaSO4:Eu, BaSrSO4, BaPbSO4, BaAl12O19:Mn, BaMgAl10O17:Eu, Zn2SiO4:Mn, (Zn,Cd)S:Ag, LaOBr, LaOBr:Tm, Lu2O2S:Eu, Lu2O2S:Tb, LuTaO4, HfO2:Ti, HfGeO4:Ti, YTaO4, YTaO4:Gd, YTaO4:Nb, Y2O3:Eu, YBO3:Eu, YBO3:Tb, and (Y,Gd)BO3:Eu.

For example, both the first light conversion layer 112 and the second light conversion layer 122 may be made of cesium iodide (CsI), and both the first light conversion layer 112 and the second light conversion layer 122 may be made of gadolinium oxysulfide (GOS). When the first light conversion layer 112 and the second light conversion layer 122 are made of the same material, the thicknesses of the first light conversion layer 112 and the second light conversion layer 122 may be different, and the light conversion characteristics of the first light conversion layer 112 and the second light conversion layer 122 may become (nearly) identical, which may facilitate the image registration and may improve the quality of the synthesized image.

In addition, at least one of the first light conversion layer 112 and the second light conversion layer 122 may also include a photoelectric material generating an electrical signal by radiation 10, and a radiation detection unit 110b or 120b having a light conversion layer 112b or 122b including a photoelectric material generating an electrical signal by radiation 10 among the first light conversion layer 112 and the second light conversion layer 122 may be the direct method of directly detecting an electrical signal generated by radiation 10 that has passed through a subject 20 by directly converting radiation 10 into an electrical signal through a photoelectric effect by the photoelectric material exhibiting photoconductive properties. Here, the photoelectric material may include selenium (Se) such as amorphous selenium (α-Se), cadmium telluride (CdTe) such as crystalloid cadmium telluride (CdTe), a crystalloid cadmium telluride compound, and the like.

Both the first radiation detection unit 110 and the second radiation detection unit 120 may be configured in the indirect method, or both may be configured in the direct method, or may be configured in a hybrid method composed of the direct-method first radiation detection unit 110b and the indirect-method second radiation detection unit 120a, or the indirect-method first radiation detection unit 110a and the direct-method second radiation detection unit 120b. In order for the first radiation detection unit 110 to detect low-energy radiation well and for the second radiation detection unit 120 to detect high-energy radiation well, the scintillator material or the photoelectric material may be selectively included in each of the first light conversion layer 112 and the second light conversion layer 122 to configure each of the first radiation detection unit 110 and the second radiation detection unit 120 in the indirect method or the direct method. Through this, the radiation detector 100 according to the present disclosure may variously configure the first radiation detection unit 110 and the second radiation detection unit 120 in an indirect-indirect, direct-direct, or direct-indirect hybrid type.

For example, when the first light conversion layer 112 (more) closer to the radiation source 210 is made of cesium iodide (CsI), since cesium iodide (CsI) has absorption characteristics for a specific wavelength band, the first light conversion layer 112 may also perform the function of a radiation energy filter, and may be configured with the first light conversion layer 112 of cesium iodide (CsI) and the second light conversion layer 122 of gadolinium oxysulfide (GOS) in a structure emphasizing the image quality of the upper layer for low energy and the sensitivity of the lower layer for low dose.

In addition, the radiation detector 100 may also be configured as a direct-indirect hybrid type, and in this case, the combination of the direct method and the indirect method may make effective separation of low-energy radiation and high-energy radiation very easy without a separate radiation energy filter.

Such a hybrid structure may enable improved spatial resolution and dose efficiency compared to what could be achieved with the direct method or the indirect method alone, and accordingly may also improve sharpness. In addition, the advantages of both the direct method and the indirect method may be taken while minimizing the respective disadvantages, the direct interaction of radiation in selenium (Se) may help maintain image sharpness, and electronic noise at high spatial frequencies may be overcome. On the other hand, the radiation signal from the scintillator (layer) may appear blurry compared to the radiation signal of amorphous selenium (α-Se), but may increase the overall detector signal with high absorption efficiency and improve low-dose performance.

That is, when the radiation detector 100 is configured as a direct-indirect hybrid type, both the first radiation detection unit 110 and the second radiation detection unit 120 may exhibit improved dose efficiency compared to using only the direct method, and may also exhibit superior spatial resolution compared to using only the indirect method, and may provide further improved quantum efficiency and improved images (particularly) for fine details and low-contrast objects through high absorption efficiency combined with high spatial resolution.

For example, among the first light conversion layer 112 and the second light conversion layer 122, the light conversion layer 112 or 122 into which radiation 10 is first incident may include a photoelectric material generating an electrical signal by radiation 10, and the light conversion layer 122 or 112 into which radiation 10 is subsequently incident may include a scintillator material converting radiation 10 into visible light. That is, by including the photoelectric material in the light conversion layer 112b or 122b of the radiation detection unit 110b or 120b (more) closer to the radiation source 210 and including the scintillator material in the light conversion layer 122a or 112a of the radiation detection unit 120a or 110a (relatively) farther from the radiation source 210, the closer radiation detection unit 110b or 120b may be configured in the direct method, and the farther radiation detection unit 120a or 110a may be configured in the indirect method. When the closer radiation detection unit 110 or 120 is configured in the direct method and the light conversion layer 112 or 122 is formed of amorphous selenium (α-Se), low-energy radiation may be detected well, and high-energy radiation may also be transmitted well. And when the farther radiation detection unit 120 or 110 is configured in the indirect method and the light conversion layer 122 or 112 is formed of gadolinium oxysulfide (GOS) having high sensitivity to high-energy radiation, high-energy radiation may be detected well. However, the present disclosure is not particularly limited thereto, and the first light conversion layer 112 and the second light conversion layer 122 may be configured in various ways.

At this time, the first light conversion layer 112 may be made of selenium (Se), and the second light conversion layer 122 may be made of gadolinium oxysulfide (GOS), and in this case, the first radiation detection unit 110 may detect low-energy radiation well by the first light conversion layer 112, and the second radiation detection unit 120 may detect high-energy radiation well by the second light conversion layer 122.

Meanwhile, the first light conversion layer 112 may be made of selenium (Se), and the second light conversion layer 122 may be made of cesium iodide (CsI). Since selenium (Se) has better sensitivity to radiation of lower (low) energy than cesium iodide (CsI), the first radiation detection unit 110 may detect relatively lower (low) energy radiation by the first light conversion layer 112 made of selenium (Se), and the second radiation detection unit 120 may detect relatively higher (low) energy radiation by the second light conversion layer 122 made of cesium iodide (CsI). Through this, the first radiation detection unit 110 and the second radiation detection unit 120 may detect radiation of mutually different energies (i.e., the first energy and the second energy).

Here, at least one of the first detection panel 111 and the second detection panel 121 may be flexible. The first detection panel 111 and the second detection panel 121 may use substrates with high heat resistance such as semiconductor substrates, quartz substrates, and glass substrates as substrates for thin film transistors (TFTs), but are not limited thereto, and may also have flexibility by using flexible substrates such as plastic, aramid, bio-nanofibers, or using flexible panels. Such flexible panels may transmit high-energy radiation well and absorb low-energy radiation well, and through this, the sensitivity of the farther radiation detection unit (for example, the second radiation detection unit) to high-energy radiation may be improved. In addition, the flexible panel may be formed thin, which may also reduce the distance between the first light conversion layer 112 and the second light conversion layer 122.

Therefore, the radiation detector 100 according to the present disclosure may reduce the distance between the first light conversion layer 112 and the second light conversion layer 122 by forming at least one of the first detection panel 111 and the second detection panel 121 as a flexible panel to make it thin, and accordingly, the reduction of radiation 10 by the first detection panel 111 and/or the second detection panel 121 may be reduced, and parallax effects such as magnification differences between the radiation image of the first energy generated through the first radiation detection unit 110 and the radiation image of the second energy generated through the second radiation detection unit 120 may also be reduced.

The radiation detector 100 according to the present disclosure may further include a support member 140 supporting at least one of the first detection panel 111 and the second detection panel 121.

The support member 140 may support at least one of the first detection panel 111 and the second detection panel 121, and may support the flexible detection panel 111 or 121. The flatness of the flexible detection panel 111 or 121 may vary depending on the object it contacts, and accordingly, the flatness of the flexible detection panel 111 or 121 may be secured through the support member 140, and the thickness of the flexible detection panel 111 or 121 may be maintained overall (or generally) uniform so that the distance between the first light conversion layer 112 and the second light conversion layer 122 may be overall constant.

The radiation detector 100 according to the present disclosure may further include a third light conversion layer 132 provided between the first detection panel 111 and the second detection panel 121.

The third light conversion layer 132 may be provided between the first detection panel 111 and the second detection panel 121, may serve as the radiation energy filter blocking low-energy (for example, the first energy) radiation and transmitting high-energy (for example, the second energy) radiation, and may also supplementarily convert low-energy radiation or high-energy radiation in the first light conversion layer 112 or the second light conversion layer 122 for use in image signal (or electrical signal) processing in the first detection panel 111 or the second detection panel 121.

At this time, the third light conversion layer 132 may be identical to at least one of the first light conversion layer 112 and the second light conversion layer 122, and may supplementarily convert radiation of the second energy or the first energy in the identical light conversion layer 122 or 112 so that the electrical signal for the image signal processing is generated. At this time, the sum of the thickness of the identical light conversion layer 122 or 112 and the thickness of the third light conversion layer 132 may be greater than or equal to the thickness of the remaining (other) light conversion layer 112 or 122.

For example, the third light conversion layer 132 may be made of the same material as the second light conversion layer 122, and may convert radiation of the second energy. Here, the second light conversion layer 122 and the third light conversion layer 132 may be light conversion layers 122 and 132 converting high-energy (or the second energy) radiation, and may be regarded as having formed the second light conversion layer 122 and the third light conversion layer 132 on the first surface and the second surface of the second detection panel 121, respectively, by dividing the thickness of the (thick) light conversion layer 122 capable of detecting high-energy radiation well from the form of making the second light conversion layer 122 (relatively) thick so as to detect high-energy radiation well. In this case, by the third light conversion layer 132 sharing the thickness of the second light conversion layer 122, sufficient (light conversion layer) thickness capable of detecting high-energy radiation well may be secured by the second light conversion layer 122 and the third light conversion layer 132, while the distance from the surface of each of the second light conversion layer 122 and the third light conversion layer 132 far from the second detection panel 121 to the second detection panel 121 may be reduced (or minimized) (compared to the thickly formed second light conversion layer). Accordingly, the light emission position(s) in the second light conversion layer 122 and the third light conversion layer 132 may become closer to the second detection panel 121, minimizing light loss, while the signal-to-noise ratio (SNR) and modulation transfer function (MTF) characteristics may be improved. At this time, the sum of the thicknesses of the second light conversion layer 122 and the third light conversion layer 132 may be greater than the thickness of the first light conversion layer 112.

FIG. 5 is a schematic cross-sectional view illustrating a radiographic apparatus according to another embodiment of the present disclosure.

The radiographic apparatus according to another embodiment of the present disclosure will be described in more detail with reference to FIG. 5, and descriptions overlapping with parts previously described in relation to the radiation detector according to one embodiment of the present disclosure will be omitted.

A radiographic apparatus 200 according to another embodiment of the present disclosure may include a radiation detector 100 according to one embodiment of the present disclosure; a radiation source 210 irradiating radiation 10 toward the radiation detector 100; and an image processing unit 220 processing the radiation of the first energy detected by the first radiation detection unit 110 and the radiation of the second energy detected by the second radiation detection unit 120 to generate a radiation image.

The radiation detector 100 may be the radiation detector 100 according to one embodiment of the present disclosure, the first radiation detection unit 110 may detect radiation of low energy (i.e., the first energy), and the second radiation detection unit 120 may detect radiation of high energy (i.e., the second energy). Through this, a dual energy radiation image may be acquired.

Here, the radiation detector 100 may (further) include a housing 150 in which the first radiation detection unit 110 and the second radiation detection unit 120 are accommodated.

The housing 150 may accommodate the first radiation detection unit 110 and the second radiation detection unit 120, and a cover portion 150a forming a radiation transmission surface may be provided (or disposed) on one surface (for example, a surface facing the radiation source). The cover portion 150a may be formed of a radiation-transmitting material, and a carbon material may be used. In addition, the housing 150 may include a back plate and a frame and/or side walls. For example, the housing 150 may be formed integrally with the back plate and the frame and/or side walls, and may be formed of an aluminum material.

The radiation source 210 may irradiate radiation 10 toward the radiation detector 100, and the radiation 10 irradiated toward the radiation detector 100 may generally be incident onto the radiation transmission surface of the first radiation detection unit 110 after passing through a subject 20, and may be detected by the first radiation detection unit 110 and the second radiation detection unit 120. Among the incident radiation 10, radiation of the first energy may be detected by the first radiation detection unit 110, and radiation of the second energy may pass through the first radiation detection unit 110 and be detected by the second radiation detection unit 120. Here, the radiation source 210 may include a collimator adjusting the irradiation area of radiation 10. The collimator may be provided in the radiation source 210, may adjust the irradiation area of radiation 10, and may also adjust the irradiation direction of radiation 10.

The image processing unit 220 may process the radiation of the first energy detected by the first radiation detection unit 110 and the radiation of the second energy detected by the second radiation detection unit 120 to generate a radiation image, and may perform subtraction and/or synthesis between the radiation image of the first energy (for example, low energy) and the radiation image of the second energy (for example, high energy) to perform image registration of the two radiation images. The image processing unit 220 may also separate bone and soft tissue using the low-energy radiation image and the high-energy radiation image, and may also improve image quality by synthesizing the radiation image of the first energy and the radiation image of the second energy.

Here, the first radiation detection unit 110 may detect radiation of the first energy, and the second radiation detection unit 120 may detect radiation of the second energy greater than the first energy. That is, since the first radiation detection unit 110 is located closer to the radiation source 210 than the second radiation detection unit 120, the first radiation detection unit 110 may detect low-energy radiation (i.e., radiation of the first energy) to acquire a low-energy radiation image, and since the second radiation detection unit 120 is located farther from the radiation source 210 than the first radiation detection unit 110, the second radiation detection unit 120 may detect high-energy radiation (i.e., radiation of the second energy greater than the first energy) to acquire a high-energy radiation image.

As such, in the present disclosure, by disposing the first radiation detection unit and the second radiation detection unit such that the second surface of the first detection panel and the second surface of the second detection panel face each other, the first detection panel and the second detection panel may have excellent thickness uniformity such that the distance between the first light conversion layer and the second light conversion layer may be generally constant, and accordingly, the image uniformity between the radiation image of the first energy generated through the first radiation detection unit and the radiation image of the second energy generated through the second radiation detection unit may be improved. At this time, by providing a light blocking layer between the first light conversion layer and the second light conversion layer, it is possible to prevent light converted by the first light conversion layer from affecting the second detection panel or light converted by the second light conversion layer from affecting the first detection panel. In addition, by forming at least one of the first detection panel and the second detection panel as a flexible panel to make it thin, the distance between the first light conversion layer and the second light conversion layer may be reduced, and accordingly, the reduction of radiation by the first detection panel and/or the second detection panel may be reduced, and parallax effects such as magnification differences between the radiation image of the first energy generated through the first radiation detection unit and the radiation image of the second energy generated through the second radiation detection unit may also be reduced.

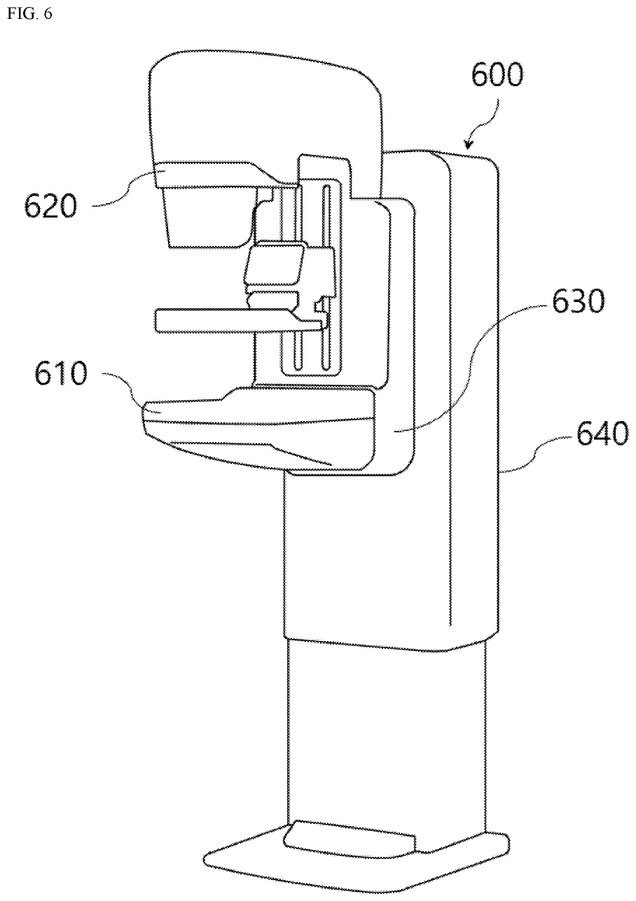

FIG. 6 illustrates a mammography apparatus according to one embodiment of the present disclosure.

When the radiation detector as described above is mounted on a mammography apparatus, a clearer image may be acquired. Unlike other X-ray apparatuses, a mammography apparatus is an apparatus that mainly images soft tissue. In addition, since the breast, which is the imaging target of the mammography apparatus, is sensitive tissue, low-dose imaging must be performed more necessarily compared to other apparatuses. In addition, in order to early detect lesions in breast tissue, it is necessary to acquire images with low noise, high contrast, and high resolution.

In this regard, conventionally, the radiation source was simply implemented at a low dose, and the insufficient parts were supplemented by image processing. However, the mammography apparatus of the present disclosure enables imaging at a lower dose than before by using a plurality of detection panels, and may acquire images of higher image quality.

In addition, as described above, when two radiation detection units are used, since a plurality of images for X-rays of two energy regions are used, the amount of image information increases, and thus a higher quality X-ray image may be acquired. More specifically, the mammography apparatus 600 of the present disclosure may acquire a synthesized image by synthesizing a plurality of radiation images based on two energies. In addition, the synthesized image acquired by the mammography apparatus 600 of the present disclosure has high resolution and noise may be significantly reduced in a specific material region. Hereinafter, the mammography apparatus 600 of the present disclosure will be described in detail.

The mammography apparatus 600 of the present disclosure may include a radiation source 620 and a radiation detector 610. The radiation source 620 may be a component irradiating radiation onto a breast of a patient. The radiation detector 610 may be disposed facing the radiation source 620. The radiation detector 610 and the radiation source 620 may be coupled to a gantry 630. The gantry 630 may rotate with respect to a column 640. The gantry 630 may rotate to image the breast from various angles. Even when the gantry 630 rotates, the detector 610 and the radiation source 620 may always face each other.

The radiation detector 610 may generate a radiation image of first energy and a radiation image of second energy based on radiation that has passed through the breast of a patient. The radiation detector 610 may further include the following components to acquire the radiation image of the first energy and the radiation image of the second energy. Since the components included in the radiation detector 610 have already been described, overlapping descriptions will be omitted. The radiation detector 610 may include a first radiation detection unit 110 and a second radiation detection unit 120.

The first radiation detection unit 110 may include a first detection panel 111 having a first surface and a second surface parallel to each other, and a first light conversion layer 112 provided on the first surface of the first detection panel 111. The first radiation detection unit 110 may generate a radiation image of first energy.

The second radiation detection unit 120 may include a second detection panel 121 having a first surface and a second surface parallel to each other, and a second light conversion layer 122 provided on the first surface of the second detection panel 121. The second radiation detection unit 120 may generate a radiation image of second energy.

The first radiation detection unit 110 and the second radiation detection unit 120 may be disposed such that the second surface of the first detection panel 111 and the second surface of the second detection panel 121 face each other.

The first radiation detection unit 110 may detect radiation of first energy from radiation incident from the first surface side of the first detection panel 111. In addition, the second radiation detection unit 120 may detect radiation of second energy from radiation that has passed through the first radiation detection unit 110 and is incident from the second surface side of the second detection panel 121. The first energy and the second energy may be different. The first energy may be lower energy than the second energy. That is, the radiation image of the first energy may be a radiation image of relatively lower energy compared to the radiation image of the second energy. The radiation image of the second energy may be a radiation image of relatively higher energy compared to the radiation image of the first energy.

At least one of the first light conversion layer 112 and the second light conversion layer 122 may include a scintillator material converting radiation into visible light. For example, the first light conversion layer 112 and the second light conversion layer 122 may include cesium iodide (CsI). However, the present disclosure is not limited thereto, and the first light conversion layer 112 and the second light conversion layer 122 may include gadolinium oxysulfide (GOS).

According to various embodiments of the present disclosure, at least one of the first light conversion layer 112 and the second light conversion layer 122 may include a photoelectric material generating an electrical signal by radiation. For example, the photoelectric material may include at least one of selenium (Se) such as amorphous selenium (α-Se), cadmium telluride (CdTe) such as crystalloid cadmium telluride (CdTe), or a crystalloid cadmium telluride compound.

According to various embodiments of the present disclosure, among the first light conversion layer 112 and the second light conversion layer 122, a light conversion layer into which radiation is first incident may include a photoelectric material generating an electrical signal by radiation. Among the first light conversion layer 112 and the second light conversion layer 122, a light conversion layer into which radiation is subsequently incident may include a scintillator material converting radiation into visible light. For example, the first light conversion layer 112 may include a photoelectric material, and the second light conversion layer 122 may include a scintillator material. However, the present disclosure is not limited thereto.

More specifically, the first light conversion layer 112 may be made of selenium (Se), and the second light conversion layer may be made of gadolinium oxysulfide (GOS). In addition, the first light conversion layer may be made of selenium (Se), and the second light conversion layer may be made of cesium iodide (CsI).

Hereinafter, a process of acquiring a synthesized image based on the radiation image of the first energy and the radiation image of the second energy acquired using the first radiation detection unit 110 and the second radiation detection unit 120 will be described.

The synthesized image is obtained by decomposing the radiation image of the first energy and the radiation image of the second energy by component, comparing the radiation image of the first energy and the radiation image of the second energy for each component, and selectively synthesizing in a direction in which sharpness and contrast increase and noise decreases. Here, the component may mean a predetermined target frequency range.

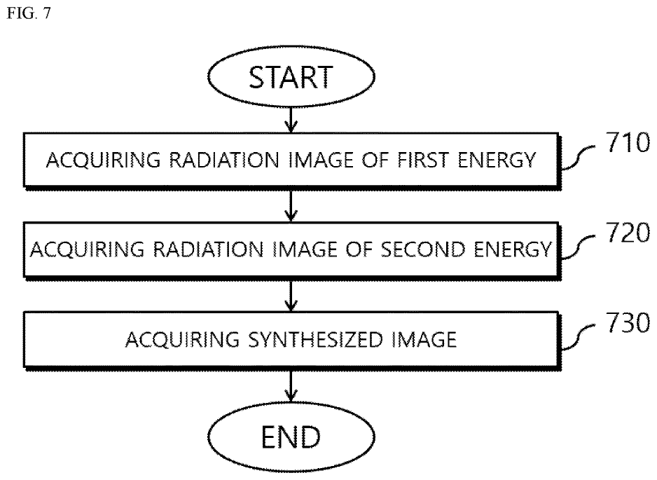

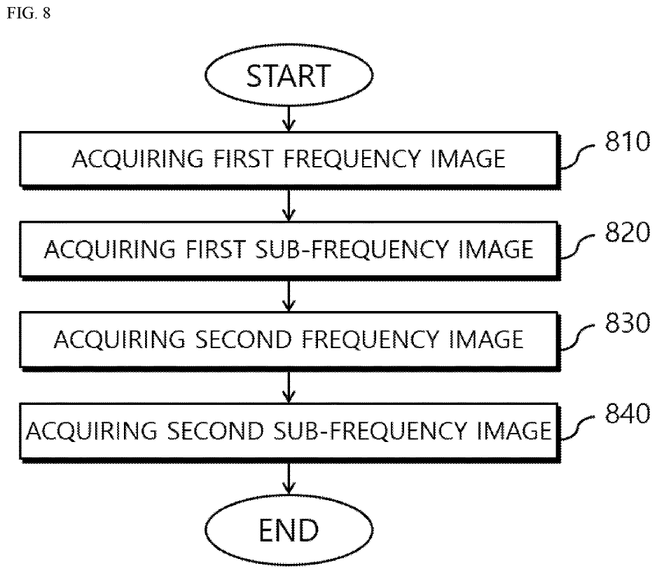

FIG. 7 is a view for describing an operation of the mammography apparatus according to one embodiment of the present disclosure.

The mammography apparatus 600 may include a controller for controlling the mammography apparatus 600. The controller may include an image processing unit 220. The controller including the image processing unit 220 may perform a step 710 of acquiring a radiation image of the first energy using the first radiation detection unit 110. In addition, the controller may perform a step 720 of acquiring a radiation image of the second energy using the second radiation detection unit 120. The controller may perform a step 730 of acquiring a synthesized image based on the radiation image of the first energy and the radiation image of the second energy.

Hereinafter, the process of acquiring the synthesized image will be described in detail.

FIG. 8 is a view for describing an operation of the mammography apparatus according to one embodiment of the present disclosure.

The controller may further perform the following steps to acquire the synthesized image.

The controller may perform a step 810 of acquiring a first frequency image by converting the radiation image of the first energy into a frequency domain. The controller may acquire the first frequency image using a frequency conversion method such as Fourier transform or Laplace transform. The controller may perform a step 820 of acquiring a first sub-frequency image for at least one predetermined target frequency range from the first frequency image. The number of first sub-frequency images may be the same as the number of target frequency ranges. One first sub-frequency image may be an image for one target frequency range. The target frequency range may be a predetermined frequency range. In this way, the controller may divide the first frequency image by target frequency range and perform different image enhancement processing for each frequency. For example, since noise characteristics may differ by frequency range, noise may be removed by different methods for each frequency range to enhance the noise removal effect.

The controller may perform a step 830 of acquiring a second frequency image by converting the radiation image of the second energy into a frequency domain. The controller may perform a step 840 of acquiring a second sub-frequency image for at least one predetermined target frequency range from the second frequency image. Since the method of acquiring the second sub-frequency image is the same as the method of acquiring the first sub-frequency image, overlapping descriptions will be omitted. The first sub-frequency image may correspond to the radiation image of the first energy, and the second sub-frequency image may correspond to the radiation image of the second energy. The first sub-frequency image and the second sub-frequency image may be images in the frequency domain.

The controller may acquire a synthesized image based on the first sub-frequency image and the second sub-frequency image.

According to one embodiment of the present disclosure, the controller may acquire a synthesized sub-frequency image by computing a weighted average of pixels at the same positions included in the first sub-frequency image and the second sub-frequency image. The weighted average may be based on the following equation.

s_pixel=w*p1+(1−w)*p2

Here, s_pixel is a value of a pixel included in the synthesized sub-frequency image, w is a weight greater than or equal to 0 and less than or equal to 1, p1 is a value of a pixel included in the first sub-frequency image, and p2 is a value of a pixel included in the second sub-frequency image. w may be a predetermined weight. In addition, the weight w may differ depending on the target frequency range. For example, a weight corresponding to a first target frequency range may differ from a weight corresponding to a second target frequency range different from the first target frequency range. The first sub-frequency image and the second sub-frequency image may be replaced with an improved first sub-frequency image and an improved second sub-frequency image.

The controller may acquire a synthesized sub-frequency image for all frequency ranges. That is, the controller may acquire at least one synthesized sub-frequency image. The controller may acquire a synthesized image by converting the synthesized sub-frequency image into a spatial domain.

According to various embodiments of the present disclosure, the controller may apply predetermined image enhancement to at least one of the first sub-frequency image and the second sub-frequency image. The controller may acquire an improved first sub-frequency image and an improved second sub-frequency image. The controller may acquire at least one improved first sub-frequency image and at least one improved second sub-frequency image for all frequency ranges.

The controller may acquire an improved first frequency image by merging at least one improved first sub-frequency image. The improved first frequency image may include the same frequency range as the first frequency image. The controller may convert the improved first frequency image into a first spatial image. The controller may acquire an improved second frequency image by merging at least one improved second sub-frequency image. The improved second frequency image may include the same frequency range as the second frequency image. The controller may convert the improved second frequency image into a second spatial image. In the above, the controller acquired the first spatial image and the second spatial image based on the improved first sub-frequency image and the improved second sub-frequency image, but the first spatial image and the second spatial image may also be acquired based on the first sub-frequency image and the second sub-frequency image. That is, the improved first sub-frequency image and the improved second sub-frequency image may not be used.

The controller may acquire a synthesized image by computing a weighted average of pixels at the same positions included in the first spatial image and the second spatial image. The weighted average may be based on the following equation.

ss_pixel=ws*ps1+(1−ws) * ps2

Here, ss_pixel is a value of a pixel included in the synthesized image, ws is a weight greater than or equal to 0 and less than or equal to 1, ps1 is a value of a pixel included in the first spatial image, and ps2 is a value of a pixel included in the second spatial image. ws may be a predetermined weight. ws may be a fixed value, or may differ depending on at least one of the values of ps1 and ps2. For example, the controller may acquire ws based on a predetermined function having ps1 and ps2 as variables.

According to one embodiment of the present disclosure, the controller may acquire a first corrected image by applying the radiation image of the first energy to an image enhancement model in order to acquire the synthesized image. The controller may acquire a second corrected image by applying the radiation image of the second energy to the image enhancement model. The controller may acquire a synthesized image based on the first corrected image and the second corrected image.

The image enhancement model may be a model for restoring a degraded image. The image generated by the mammography apparatus 600 may be degraded by light scattering due to physical characteristics inherent to the indirect-method detector, x-ray scattering due to a thick subject, and the like. Therefore, the controller may restore the degraded image using the image enhancement model for restoring the degraded image. The image enhancement model may be a predetermined function having pixel values included in at least one of the radiation image of the first energy and the radiation image of the second energy as variables. The controller may acquire at least one of the first corrected image or the second corrected image based on the image enhancement model. In addition, the controller may acquire a synthesized image based on the first corrected image and the second corrected image.

The controller may apply the image enhancement model to the radiation image of the first energy to acquire the first sub-frequency image. More specifically, the controller may acquire a first corrected image by applying the radiation image of the first energy to the image enhancement model. The image enhancement model may be processed in the spatial domain rather than the frequency domain. The controller may perform a step 810 of acquiring a first frequency image by converting the first corrected image into the frequency domain. The controller may perform a step 820 of acquiring a first sub-frequency image for at least one predetermined target frequency range from the first frequency image.

However, the present disclosure is not limited thereto, and the image enhancement model may also be applied in the frequency domain. For example, the controller may perform a step 810 of acquiring a first frequency image by converting the radiation image of the first energy into the frequency domain. The controller may acquire a first restored frequency image by applying the image enhancement model to the first frequency image. The controller may perform a step 820 of acquiring a first sub-frequency image for at least one predetermined target frequency range from the first restored frequency image.

In addition, the controller may apply the image enhancement model to the radiation image of the second energy to acquire the second sub-frequency image. More specifically, the controller may acquire a second corrected image by applying the radiation image of the second energy to the image enhancement model. The image enhancement model may be processed in the spatial domain rather than the frequency domain. The controller may perform a step 830 of acquiring a second frequency image by converting the second corrected image into the frequency domain. The controller may perform a step 840 of acquiring a second sub-frequency image for at least one predetermined target frequency range from the second frequency image.

However, the present disclosure is not limited thereto, and the image enhancement model may also be applied in the frequency domain. For example, the controller may perform a step 830 of acquiring a second frequency image by converting the radiation image of the second energy into the frequency domain. The controller may acquire a second restored frequency image by applying the image enhancement model to the second frequency image. The controller may perform a step 840 of acquiring a second sub-frequency image for at least one predetermined target frequency range from the second restored frequency image.

The controller may acquire a synthesized image based on the first sub-frequency image and the second sub-frequency image. Since the process of acquiring the synthesized image has already been described, overlapping descriptions will be omitted.

The image enhancement model may operate as follows.

A method of processing a radiation image according to the image enhancement model of the present disclosure may include acquiring a radiation image including at least one of a radiation image of first energy or a radiation image of second energy using an indirect-method radiation detector including light conversion layers 112 and 122 and detection panels 111 and 121. The method of processing a radiation image according to the image enhancement model may include determining a value of a parameter defined in a point spread function (PSF) according to the light conversion layers 112 and 122 or the detection panels 111 and 121. In addition, the method of processing a radiation image according to the image enhancement model may include acquiring a corrected image including at least one of a first corrected image or a second corrected image by deconvolving and correcting at least one of the radiation image of the first energy or the radiation image of the second energy using the point spread function (PSF) to which the value of the parameter has been applied.

When a radiation image including at least one of the radiation image of the first energy or the radiation image of the second energy is captured with an indirect-method radiation detector using the light conversion layers 112 and 122, light is scattered by the light conversion layers 112 and 122, causing a blur phenomenon in the radiation image. At this time, the scattering of light by the light conversion layers 112 and 122 may have a Gaussian form. In order to correct such a blur phenomenon of the radiation image and improve the sharpness of the radiation image, the present disclosure defines the radiation image including the blur phenomenon as an image obtained by convolving a clear radiation image with a point spread function (PSF), and corrects the radiation image by deconvolving the radiation image including the blur phenomenon using the point spread function (PSF).

The image enhancement model may be applied to images acquired with an indirect-method radiation detector. That is, when the radiation image of the first energy is acquired by the direct method and the radiation image of the second energy is acquired by the indirect method, the controller may apply the image enhancement model to the radiation image of the second energy. As already described, the indirect method may use a scintillator material and the direct method may use a photoelectric material.

The detection panels 111 and 121 may store, for each pixel, charges generated by visible light converted when radiation is irradiated onto the light conversion layers 112 and 122. Here, the resolution of the radiation image may differ depending on the pixel size of the detection panels 111 and 121.

In addition, the value of the parameter defining the point spread function (PSF) may be determined according to the light conversion layers 112 and 122 or the detection panels 111 and 121 in parallel with the step of acquiring the radiation image. The step of determining the value of the parameter may be performed simultaneously with the step of acquiring the radiation image, may be performed before the step of acquiring the radiation image, or may be performed after the step of acquiring the radiation image. The controller may determine the value of the parameter defining the point spread function (PSF) according to the light conversion layers 112 and 122 or the detection panels 111 and 121. At this time, the controller may determine the value of the parameter defining the point spread function (PSF) according to the pixel size of the detection panels 111 and 121, the thickness of the light conversion layers 112 and 122, and the type of scintillator material included in the light conversion layers.

In the step of determining the value of the parameter, the value of the parameter may be determined as a value of the parameter for which the corrected image obtained by deconvolving the radiation image using the point spread function (PSF) satisfies an image quality criterion. When the radiation image is deconvolved and corrected, the sharpness of the corrected image is improved beyond the sharpness of the radiation image, but if the sharpness is improved too artificially, the inherent characteristics of the radiation image are distorted. Since the inherent characteristics of the radiation image lose their value as a medical diagnostic image when distorted, it is possible to filter out corrected images in which the inherent characteristics of the radiation image are distorted by evaluating the image quality of the corrected image and determining whether the inherent characteristics of the radiation image are distorted. Therefore, when the value of the parameter is determined as a value of the parameter satisfying the image quality criterion, the radiation image may be corrected with a corrected image having improved sharpness without distorting the inherent characteristics of the radiation image.

The image quality may be evaluated by measuring at least one selected from a modulation transfer function (MTF), detective quantum efficiency (DQE), normalized noise power spectrum (NNPS), and signal-to-noise ratio (SNR). The measured values of the modulation transfer function (MTF), detective quantum efficiency (DQE), normalized noise power spectrum (NNPS), and signal-to-noise ratio (SNR) may be measured using an image evaluation tool, and the image quality may be evaluated by analyzing a characteristic graph of each measured value. That is, whether the inherent characteristics of the radiation image are distorted may be determined through the characteristic graph of each measured value.

The modulation transfer function (MTF) represents the frequency response of an image system, and may be defined as the ratio of image contrast to subject contrast, which is a measurement factor related to sharpness. In addition, the modulation transfer function (MTF) measures the spatial frequency of an image as the frequency recording capability of a region corresponding to each frequency, indicates the degree to which the image system responds to spatial changes in the image to determine resolution, is widely used to evaluate the sharpness of an image, and may be used to determine whether sharpness has been improved through the modulation transfer function (MTF).

The detective quantum efficiency (DQE) represents the transfer characteristics of the signal-to-noise ratio (SNR), and is a parameter expressing the amount of noise produced by the final image. That is, it measures the overall signal-to-noise ratio performance of the radiation detector and the ability of the radiation detector to transfer the signal-to-noise ratio. In addition, the detective quantum efficiency (DQE) may be defined as the square of the ratio of the output signal-to-noise ratio (SNR) to the input signal-to-noise ratio (SNR).

In addition, since the detective quantum efficiency (DQE) is a comprehensive indicator representing the quality of a radiation image and is derived from the modulation transfer function (MTF), normalized noise power spectrum (NNPS), signal-to-noise ratio (SNR), and the like, image quality may be comprehensively evaluated including the modulation transfer function (MTF), normalized noise power spectrum (NNPS), and signal-to-noise ratio (SNR), and whether the inherent characteristics of the image are distorted may be effectively determined.

The noise power spectrum (NPS) expresses the distribution of variance values of noise on the spatial frequency, represents the dependence of noise, which is a variation factor between pixels in an image, on spatial frequency, and is a noise-related measurement factor. In addition, the normalized noise power spectrum (NNPS) refers to the normalized noise power spectrum (NPS), and may be obtained by averaging samples of all spectra.

Noise in an image means uncertainty or inaccuracy in an image signal, and may be broadly classified into noise attributable to the number of photons constituting image information and noise due to an image signal processing circuit. In the former, when the number of photons constituting image information is small, the degree of uncertainty increases, whereas when the number of photons increases, the probability of being detected as an image signal increases, and the influence of noise on the image signal may decrease. In order to evaluate the signal-to-noise ratio of a radiation image system, an image may be acquired using a lead phantom from which the central portion has been removed, and the gray-scale values of the acquired image measured along a reference line may be used. The image signal means the difference (delta_D) in gray-scale values between the surrounding lead phantom and the central circular region, and the image noise means the standard deviation(s) of the gray-scale values of the acquired image, and the ratio thereof (delta_D/s) may be defined as the signal-to-noise ratio (SNR), and this signal-to-noise ratio (SNR) is a measurement factor related to signal and noise.

The controller may acquire a corrected image by deconvolving the radiation image using the point spread function (PSF) to which the value of the parameter has been applied. The radiation image is deconvolved and corrected using the point spread function (PSF) to which the value of the parameter has been applied, and if only the value of the parameter is known, the radiation image may be corrected by deconvolving the radiation image with the point spread function (PSF). The deconvolution process assumes that an image including a blur phenomenon is generated by convolution of a point spread function (PSF) and a clear image, and may iteratively perform an algorithm until the residual error value between a blur image obtained from imaging (i.e., including light scattering by a scintillator) and a blur image estimated by a user is sufficiently small. In addition, the deconvolution may use spatial-invariant deconvolution that restores the entire image using a single point spread function (PSF). Meanwhile, spatial-variant deconvolution that restores an image using a plurality of individual point spread functions (PSFs) depending on the position within the radiation image may also be used, and although spatial-variant deconvolution using individual point spread functions (PSFs) appropriate for each position may have higher sharpness than spatial-invariant deconvolution, spatial-invariant deconvolution may be mainly used because the process of obtaining individual point spread functions (PSFs) appropriate for each position is complex.

The indirect-method radiation detector has higher detective quantum efficiency (DQE) than the direct-method radiation detector, but has lower sharpness (or MTF) than the direct-method radiation detector. Here, the detective quantum efficiency (DQE) is a radiation conversion efficiency, and a higher detective quantum efficiency (DQE) means that a good image may be obtained even with a lower radiation dose. Therefore, in the present disclosure, when using an indirect-method radiation detector, scintillator scatter occurring from the structural characteristics of hardware is corrected by a software algorithm, making it possible to obtain high sharpness comparable to a direct-method radiation detector while maintaining the high detective quantum efficiency (DQE) of the indirect-method radiation detector.

As a result of performing the image enhancement model according to the present disclosure, an image having high sharpness and higher detective quantum efficiency (DQE) than a direct-method radiation detector could be obtained, and in this case, there is an effect of obtaining a clearer and higher quality image with a lower radiation dose. That is, when a product with high detective quantum efficiency (DQE) is used, less radiation dose needs to be irradiated, thereby reducing the patient's radiation exposure dose, and when sharpness is high, a clearer image may be obtained, which is useful for diagnosis.

The controller may use a mathematical noise model to reduce noise in the synthesized image. In general, it is difficult to effectively remove only noise components from an image, and an excessive noise filter may risk making the image blurry. An AI noise model based on the noise characteristics of X-ray images and the information of the two images acquired by the detector of the present disclosure (the radiation image of the first energy or the radiation image of the second energy) enables more accurate identification of noise components with more information compared to a single-energy image, making it possible to stably remove noise components.

The controller may acquire noise information by applying at least one of the radiation image of the first energy or the radiation image of the second energy to a noise model. The controller may acquire an improved synthesized image from which noise is removed from the synthesized image based on the noise information.

The noise model may be a model extracting noise information based on at least one of the radiation image of the first energy or the radiation image of the second energy. The noise model may be a rule-based model or a machine learning model. The noise model may be a model that has machine-learned the relationship of noise information with respect to the radiation image of the first energy or the radiation image of the second energy.

According to various embodiments of the present disclosure, the controller may acquire noise information by applying at least one of the first sub-frequency image or the second sub-frequency image to the noise model. The controller may acquire at least one of an improved first sub-frequency image or an improved second sub-frequency image by removing noise information acquired from at least one of the first sub-frequency image or the second sub-frequency image, and may acquire a synthesized image based on the improved first sub-frequency image or the improved second sub-frequency image. Since the process of acquiring the synthesized image based on the first sub-frequency image or the second sub-frequency image has already been described, overlapping descriptions will be omitted.

The mammography apparatus 600 using the method of acquiring a synthesized image of the present disclosure may acquire images of an equivalent level to images acquired at a high dose even at a low dose. Since images of different X-ray energies for the same subject are acquired, contrast is improved compared to single-energy images, and the influence of artifacts may be minimized using a mathematical model based on physical laws.

Although preferred embodiments of the present disclosure have been illustrated and described above, the present disclosure is not limited to the above embodiments, and those skilled in the art will understand that various modifications and equivalent other embodiments are possible therefrom without departing from the gist of the present disclosure as claimed in the claims. Therefore, the technical protection scope of the present disclosure should be determined by the claims below.

What is claimed is:

1. A mammography apparatus comprising:
a radiation source irradiating radiation onto a breast of a patient; and
a radiation detector disposed facing the radiation source, the radiation detector generating a radiation image of first energy and a radiation image of second energy based on radiation that has passed through the breast of the patient,
wherein the radiation detector comprises:
a first radiation detection unit comprising a first detection panel having a first surface and a second surface parallel to each other, and a first light conversion layer provided on the first surface of the first detection panel, the first radiation detection unit generating the radiation image of the first energy; and
a second radiation detection unit comprising a second detection panel having a first surface and a second surface parallel to each other, and a second light conversion layer provided on the first surface of the second detection panel, the second radiation detection unit generating the radiation image of the second energy,
wherein the first radiation detection unit and the second radiation detection unit are disposed such that the second surface of the first detection panel and the second surface of the second detection panel face each other.

2. The mammography apparatus of claim 1, wherein:
the first radiation detection unit detects radiation of first energy from radiation incident from a first surface side of the first detection panel,
the second radiation detection unit detects radiation of second energy from radiation that has passed through the first radiation detection unit and is incident from a second surface side of the second detection panel, and
the first energy and the second energy are different.

3. The mammography apparatus of claim 1, wherein at least one of the first light conversion layer and the second light conversion layer comprises a scintillator material converting radiation into visible light.

4. The mammography apparatus of claim 3, wherein the first light conversion layer and the second light conversion layer are made of cesium iodide (CsI).

5. The mammography apparatus of claim 3, wherein the first light conversion layer and the second light conversion layer are made of gadolinium oxysulfide (GOS).

6. The mammography apparatus of claim 1, wherein at least one of the first light conversion layer and the second light conversion layer comprises a photoelectric material generating an electrical signal by radiation.

7. The mammography apparatus of claim 1, wherein:
among the first light conversion layer and the second light conversion layer, a light conversion layer into which radiation is first incident comprises a photoelectric material generating an electrical signal by the radiation, and
among the first light conversion layer and the second light conversion layer, a light conversion layer into which the radiation is subsequently incident comprises a scintillator material converting the radiation into visible light.

8. The mammography apparatus of claim 7, wherein:
the first light conversion layer is made of selenium (Se), and
the second light conversion layer is made of gadolinium oxysulfide (GOS).

9. The mammography apparatus of claim 7, wherein:
the first light conversion layer is made of selenium (Se), and
the second light conversion layer is made of cesium iodide (CsI).

10. The mammography apparatus of claim 1, comprising a controller for controlling the mammography apparatus,
wherein the controller acquires the radiation image of the first energy using the first radiation detection unit,
the controller acquires the radiation image of the second energy using the second radiation detection unit, and
the controller acquires a synthesized image based on the radiation image of the first energy and the radiation image of the second energy.

11. The mammography apparatus of claim 10, wherein the controller:
in order to acquire the synthesized image,
acquires a first corrected image by applying the radiation image of the first energy to an image enhancement model,
acquires a second corrected image by applying the radiation image of the second energy to the image enhancement model, and
acquires the synthesized image based on the first corrected image and the second corrected image.

12. The mammography apparatus of claim 10, wherein the controller:
acquires noise information by applying at least one of the radiation image of the first energy or the radiation image of the second energy to a noise model, and
acquires an improved synthesized image from which noise is removed from the synthesized image based on the noise information.

13. The mammography apparatus of claim 10, wherein the radiation image of the first energy is a radiation image of lower energy compared to the radiation image of the second energy.
